(19) United States
(12) Patent Application Publication
(10) Pub. No.: US 2021/0017591 A1
(43) Pub. Date: Jan. 21, 2021

(54) METHOD FOR SELECTION OF CORRECT NUCLEIC ACIDS

(71) Applicant: Carl Philipp Dodo Christian Graf zu Innhausen und Knyphausen, Cologne (DE)

(72) Inventor: Carl Philipp Dodo Christian Graf zu Innhausen und Knyphausen, Cologne (DE)

(21) Appl. No.: 16/946,376

(22) Filed: Jun. 18, 2020

Related U.S. Application Data
(60) Provisional application No. 62/865,322, filed on Jun. 24, 2019.

(51) Int. Cl. C12Q 1/6853

(52) U.S. Cl. CPC C12Q 1/6853

(57) ABSTRACT

Selective removal of erroneous nucleic acids or the selective retrieval of correct nucleic acids is enabled by controlled complementary strand synthesis using compositions of nucleotides at each cycle of the synthesis that facilitate the extension of correctly templated complementary strands and the termination of incorrectly templated complementary strands to the effect of allowing sufficient biochemical discrimination between correct and erroneous nucleic acids, for example, based on the completeness of the complementary strand synthesis.

### BACKGROUND OF THE INVENTION

Field of the Invention: The present invention describes a method for selective retrieval of correct nucleic acids or removal of incorrect nucleic acids, for example in the field of artificial synthesis of DNA or other nucleic acids.

Description of the Related Art: There is a growing demand for de novo synthesised double-stranded or single-stranded DNA, RNA or XNA. Target sequences of synthetic DNA, RNA or XNA may be of artificial or natural origin or any combination of natural and artificial origin and may be entirely predefined or partially or fully degenerate.

Methods for the synthesis for said molecules are well known in the art. One example of such a synthesis method is a chemical process involving specialised phosphoramidite coupling chemistry and cycles of activation, coupling and deprotection, whereby the sequence of the growing synthetic nucleic acid is controlled by providing certain desired nucleotides at each cycle of said cyclical synthesis process. Another example is the use of template-independent or template-dependent nucleic acid polymerase enzymes (e.g. terminal nucleotidyl transferases [SEQ ID NO:10] and DNA polymerases [SEQ ID NO:2] in combination with a universal base template, respectively), which may be provided with particular desired modified or unmodified nucleotides at a given step or cycle of the synthesis process to control the incorporation of said nucleotides. Longer synthetic nucleic acids (polynucleotides) are typically assembled from shorter synthetic single-stranded nucleic acid fragments (oligonucleotides) by designing overlapping regions of sequences in such a way that multiple fragments are likely to hybridise in the correct order when they are annealed under appropriate conditions.

The synthesis methods described above suffer from certain inefficiencies, for example in the cyclical process of nucleotide coupling, leading to the undesired production of erroneous nucleic acid fragments. These errors can be of different type depending on whether a nucleotide failed to be incorporated or an additional nucleotide or wrong nucleotide is incorporated, which leads to deletion, insertion or substitution errors, respectively. Since errors occur at a certain rate at each cycle of the nucleic acid synthesis, the fraction of erroneous strands of nucleic acids in the synthesis pool scales with the number of cycles (i.e. the fragment length) according to a power law. With current state-of-the-art synthesis methods, synthesis lengths of approximately 200 base pairs (bp) are typically the limit at which correct molecules can be retrieved in reasonable quantities, which makes it necessary to assemble fragments exceeding this length from multiple fragments in the hybridisation process described above. As each oligonucleotide is produced with a certain error rate, a significant fraction of nucleic acids resulting from said assembly processes will contain errors as well and the chance of incorporating at least one error increases with the number of oligonucleotides being used for hybridisation.

Some methods to detect or reduce errors are known in the art. Synthesised oligonucleotides may be subject to certain purification procedures (e.g. high performance liquid chromatography or polyacrylamide gel electrophoresis), which allows for enrichment of error-free molecules based on length or charge. However, said purification methods are usually expensive, imperfect and not suitable for detecting substitution errors. Moreover, said purification methods are inherently incompatible with highly parallelised production of oligonucleotides (e.g. microarray- or microchip-based oligonucleotide synthesis), as they require detachment and elution from the solid support and typically require too large initial synthesis quantities.

Another method for detection or removal of erroneous nucleic acids involves molecular cloning of double-stranded nucleic acids (for example obtained from hybridisation processes described above). In molecular cloning, the manufactured nucleic acid molecules are provided to host cells (e.g. a bacteria) under conditions where singular uptake of a randomly selected nucleic acid molecule into the host cell is likely and where said molecule is then copied multiple times in a monoclonal manner. By isolating single colonies of bacteria and performing sequencing of the monoclonally copied nucleic acids, an error-free nucleic acid can be identified from the synthesis pool. However, this cloning based method is relatively expensive, slow and ultimately limited by the initial yield of correct nucleic acids.

Other error correction methods of nucleic acids known in the art are based one the detection of mismatched regions in hybridised regions of at least two complementary nucleic acids. One common way to detect said mismatches in hybridised fragments is by using one or more mismatch detecting enzymes or binding domains. Examples for mismatch-specific enzymes are Escherichia coli endonuclease V [SEQ ID NO:3], T4 endonuclease VII [SEQ ID NO:4] and T7 endonuclease I [SEQ ID NO:5]. The reaction of these enzymes with mismatched hybridised nucleic acids may leave single-stranded overhangs, which can be targeted for degradation by proofreading DNA polymerases [SEQ ID NO:2] or single-strand-specific exonucleases [SEQ ID NO:6]. By performing hybridisation reactions in a hierarchical manner (starting from oligonucleotides) and including error detection procedures at multiple or all hybridisation steps, the yield of error-free target nucleic acids can be greatly improved. A limitation of the above error detection/correction methods is that at least one of two single-stranded nucleic acids used for hybridisation needs to be eluted from the solid support used for the initial synthesis, which makes it necessary to compartmentalise the respective fragments to be hybridised or to have precise fluid control of the solutions carrying the respective fragments. Both compartmentalisation and fluid control are difficult to achieve on the scales of microchips/-arrays used for highly parallelised and automated oligonucleotide synthesis.

Methods described herein include “uncontrolled” or “controlled” complementary strand synthesis. “Uncontrolled” complementary strand synthesis refers to enzymatic template-dependent nucleic acid polymerization under provision of nucleotides that allow said polymerization to continue until the 5′-end of the template strand or another endpoint for the polymerase is reached. “Controlled” complementary strand synthesis refers to a cyclical method of enzymatic template-dependent nucleic acid polymerization under provision of nucleotides at each cycle of said cyclical method that allow said polymerization only to continue for a limited number of positions of the template.

Methods described herein may include the use “reversibly terminated” nucleotides or “reversible terminators” and the use of “irreversibly terminated” nucleotides or “irreversible terminators”. Reversibly terminated nucleotides or reversible terminators are nucleotides that block polymerization after being incorporated into a nucleic acid strand but that may be “unblocked” or “deprotected” to allow further extension of said nucleic acid. Reversibly terminated nucleotides or reversible terminators are well known in the art, for example, in the field of high-throughput DNA sequencing technology based on sequencing by synthesis.

Examples of reversibly terminated nucleotides or reversible terminators are 3′-O-blocked reversible terminators where the blocking group is linked to the oxygen atom of the 3′-OH of the pentose or 3′-OH unblocked reversible terminators also known as “virtual terminators” where an blocking group is linked to, for example, the base. Blocking groups may be labile under certain conditions to allow for their removal, referred to as “unblocking” or “deprotection”.

For example, unblocking or deprotection may be achieved through heating above a certain temperatures, changes in pH or electromagnetic irradiation. Reversibly terminated nucleotides or reversible terminators may bear further modifications such as fluorescent groups that allow detection of their incorporation into a nucleic acid. Specifically engineered or evolved polymerase enzymes [SEQ ID NO:7] may be used that more readily accept nucleotides with said blocking groups or fluorescent groups.

Irreversibly terminated nucleotides or irreversible terminators are nucleotides that permanently block polymerization after being incorporated into a nucleic acid strand. Irreversibly terminated nucleotides or irreversible terminators are well known in the art, for example, in the field of Sanger DNA sequencing technology. An examples of irreversibly terminated nucleotides or irreversible terminators are dideoxy-nucleotides where termination happens due the lack of a 3′-OH group at the pentose. Irreversibly terminated nucleotides or irreversible terminators may bear further modifications such as fluorescent groups that allow detection of their incorporation into a nucleic acid.

### SUMMARY

The present invention describes methods for the selective removal of erroneous nucleic acids or the selective retrieval of correct nucleic acids enabled by controlled complementary strand synthesis using compositions of nucleotides at each cycle of the synthesis that facilitate the extension of correctly templated complementary strands and the termination of incorrectly templated complementary strands to the effect of allowing sufficient biochemical discrimination between correct and erroneous nucleic acids, for example, based on the completeness of the complementary strand synthesis. In one embodiment of the invention, each cycle of said complementary strand synthesis comprises the provision of mixtures of modified nucleotides aiming at extending those complementary strands which present an expected correct template base at the interrogated position with a corresponding reversibly terminated nucleotide and those complementary strands which present any incorrect template base at the interrogated position with a corresponding irreversibly terminated nucleotide.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts an overview of one example of a workflow according to various embodiments of the invention.

FIG. 2 illustrates one example cycle of controlled complementary strand synthesis according to various embodiments of the invention described herein, detailing the use of a mixture of reversibly terminated nucleotides and irreversibly terminated nucleotides.

FIG. 3 illustrates one example cycle of controlled complementary strand synthesis according to various embodiments of the invention described herein, detailing the use of reversibly terminated nucleotides and capping of unextended 3′OH groups.

FIG. 4 illustrates example cycles of controlled complementary strand synthesis according to various embodiments of the invention described herein, detailing the use of natural nucleotides.

FIG. 5 depicts an overview of one example of a workflow according to various embodiments of the invention, detailing immobilisation of nucleic acids at the 5′ end.

FIG. 6 depicts an overview of one example of a workflow according to various embodiments of the invention, detailing immobilisation of nucleic acids at the 5′ end and the use of hairpin loops for self-priming of complementary strand synthesis.

FIG. 7 depicts one example of a workflow to retrieve error-free nucleic acids after controlled complementary strand synthesis according to various embodiments of the invention, detailing the use of a double-strand specific restriction enzyme [SEQ ID NO:8] for the release from a solid support.

FIG. 8 depicts one example of a workflow to retrieve error-free nucleic acids after controlled complementary strand synthesis according to various embodiments of the invention, detailing the use of denaturing conditions to release complementary strands from a solid support and targeted degradation of single-stranded nucleic acids originating from erroneous strands.

FIG. 9 depicts one example of a workflow to retrieve error-free nucleic acids after controlled complementary strand synthesis according to various embodiments of the invention, detailing a cyclical process in which denaturing conditions are used to release complementary strands from a solid support followed by an optional amplification step, binding onto a solid support based on a primer binding site only present in previously completely synthesized strands and subsequent controlled complementary strand synthesis.

FIG. 10 depicts one example of a workflow to retrieve error-free nucleic acids after controlled complementary strand synthesis according to various embodiments of the invention, detailing the use of denaturing conditions to release complementary strands from a solid support and polymerase chain reaction-based amplification of error-free nucleic acids.

FIG. 11 depicts one example of a workflow to selectively remove erroneous nucleic acids from a solid support after controlled complementary strand synthesis according to various embodiments of the invention, detailing 5′ end immobilised nucleic acids and the use of single-strand specific endonucleases [SEQ ID NO:9].

FIG. 12 depicts one example of a workflow to selectively remove erroneous nucleic acids from a solid support after controlled complementary strand synthesis according to various embodiments of the invention, detailing 3′ end immobilised nucleic acids, 5′ protected ends and the use of single-strand specific endonucleases [SEQ ID NO:9] and 5′ specific exonucleases [SEQ ID NO:1].

FIG. 13 depicts one example of a workflow according to various embodiments of the invention, detailing the use of strand-displacing polymerases.

### DETAILED DESCRIPTION

Nucleic acids of various sources may be subjected to the technique described herein and, despite using DNA as an example of a nucleic acid for the subsequent description, it will be appreciated that the technique could also be applied to other types of nucleic acid, such as XNA (xeno nucleic acid) or RNA.

The technique described herein provides a method of retrieving or enriching for error-free DNA from a mixture of erroneous and error-free DNA, which may be a product of de novo nucleic acid synthesis or other processes and which may contain any type of errors such as deletions (where one or multiple nucleotides are missing), insertions (where one or multiple additional nucleotides are inserted), substitutions (where one or multiple nucleobases are exchanged for other nucleobases) or chemical alterations of the structure of the nucleic acid.

The technique described herein addresses the demand for accurately synthesized DNA in various technological field, such as biotechnology, nanotechnology or data storage and avoids expensive and time-consuming techniques, such as molecular cloning, hybridisation-based error correction or barcode-based retrieval of sequence-confirmed DNA.

In the present invention, retrieval of or enrichment for error-free DNA from a heterogeneous population of error-free and erroneous DNA is enabled by controlled complementary strand synthesis. Since specific compositions of nucleotides are provided at each cycle of the synthesis according to the expected sequence of the interrogated template strand, only templates that present the correct template base at each cycle (or in case of deletions or insertions in homopolymer regions the correct number of the same base in a row) will ultimately be able to follow this “dictated” synthesis while an erroneous template strand will fail the complete synthesis of its corresponding complementary strand. The resulting difference between error-free and erroneous templates in the success of complementary strand synthesis can then be leveraged for selective degradation or inactivation of erroneous strands or selective amplification or elution of error-free strands.

Use of this method may be envisioned at multiple stages in the process of DNA synthesis, for example at the stage of short oligonucleotide synthesis or after the stage of fragment assembly from short oligonucleotides. The method is not limited to any specific synthesis method or source of nucleic acid, for example it may be applied in the currently most common nucleic acid synthesis technique, phosphoramidite-based oligonucleotide synthesis, or in enzymatic synthesis techniques based on terminal deoxynucleotidyl transferases [SEQ ID NO:10].

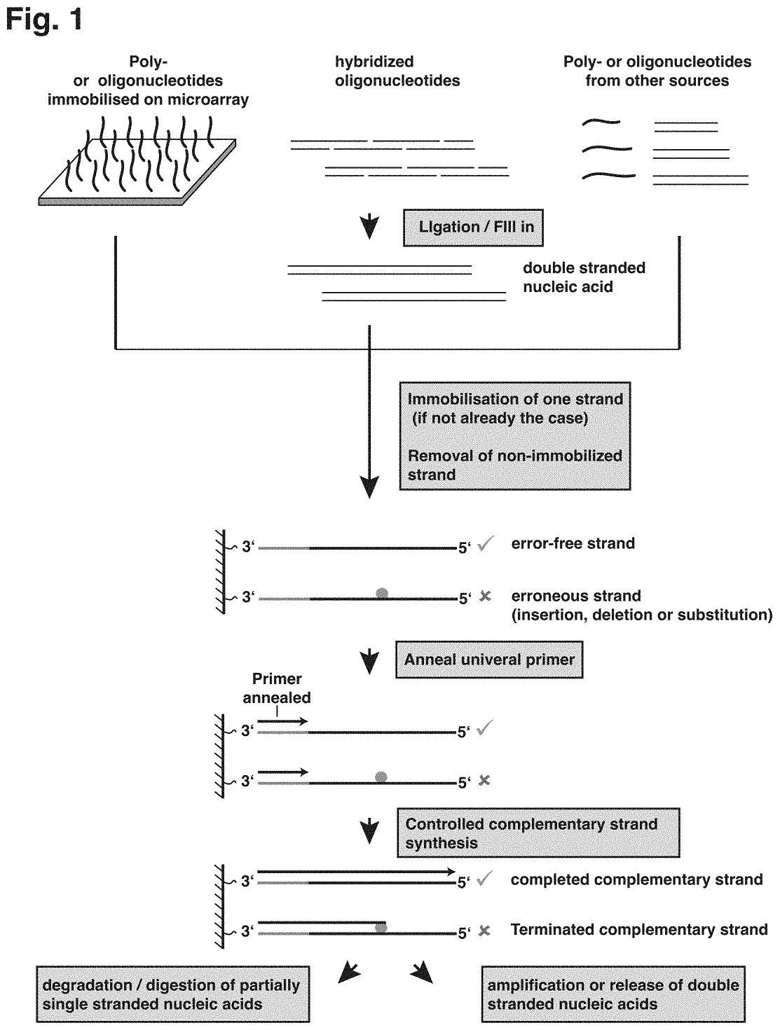

FIG. 1 schematically illustrates an example of a workflow of the method described herein. Starting from various sources of nucleic acids (e.g. microarray-based oligonucleotide synthesis or longer assemblies of DNA), which may be immobilized on a solid support and which, for double-stranded DNA, may undergo treatment to remove one of the two strands. At the next step, one error-free and one erroneous strand are shown schematically, with each respective 3′ end immobilized on a solid support and with a small circle indicating an error of any of the types mentioned above. It should be obvious that in reality many molecules will be present at once and, moreover, for example in parallelized synthesis approaches such as microarray-based oligonucleotide synthesis, different target oligonucleotides may be present on different spots of a chip with a certain spatial patterning and means to address each spot individually (e.g. through differential illumination, differential temperature regulation or differential control of acidity). The immobilized nucleic acids which will serve as synthesis templates are shown with an annealed primer at the next step of the flowchart, the binding site of which may be present on all immobilized template strands. Alternatively, a range of different primer sequences may be used, for example to allow for interrogation of nucleic acids on certain spots of a microarray. At the next step an exemplified output of the controlled complementary strand synthesis is shown, which, in the illustrated case, led to a completed complementary strand for the error-free template strand and to an incompletely synthesized complementary strand for the erroneous template due to termination at the site of the error, leaving the template strand partially single-stranded. FIG. 1 indicates that the output of said complementary strand synthesis may be further processed to selectively degrade erroneous and thus incompletely synthesized strands or to selectively amplify or release error-free and thus completely synthesized strands. The workflow in FIG. 1 may be controlled partially or completely with control devices, such as computer programs, fluid control system and apparatuses to address individual spots of immobilized nucleic acids as mentioned above.

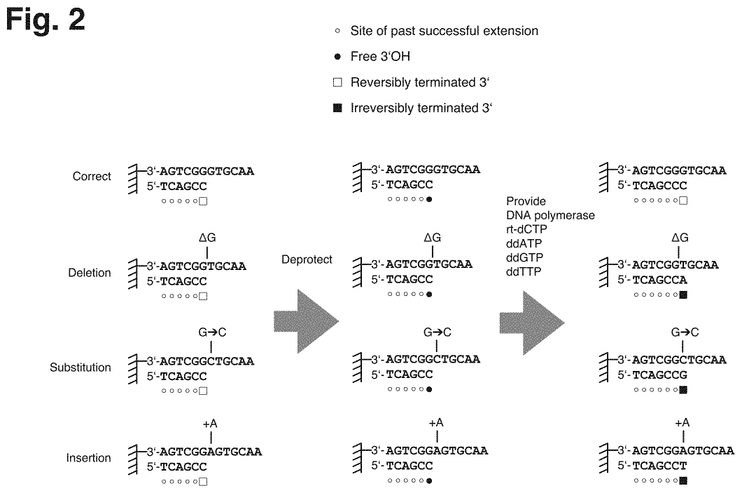

A detailed view of one cycle of said controlled complementary strand synthesis according to a preferred embodiment of the present invention is shown in FIG. 2. For clarity, a very short stretch of DNA sequence is shown [SEQ ID NO:14-18], although obviously the method is not limited to short DNA sequences. At the beginning of said cycle, all complementary strands shown (error-free and three erroneous strands each with a different type of error) have been extended by six nucleotides in previous cycles according to the 3′-immobilized template strand and, at this point, further extension of all strands is reversibly blocked by protection moieties, for example by the presence of a modification at the terminal nucleotide or because of the use of single-turnover variants of DNA polymerases, which sterically or chemically block further extension at the terminal nucleotide. Continuation of complementary strand synthesis thus requires a deprotection step, which, in the example, leads to the generation of a free 3′OH at the terminal nucleotides as an acceptor site for the next nucleotide. After deprotection and exposure of 3′OH groups at the termini, a mixture of modified nucleotides is provided, for example through a fluidic control system. In the example, a ‘G’ is expected at the interrogated site in the template strand and thus reversibly terminated dCTP (rt-dCTP) is provided together with a DNA polymerase, leading to extension of the correctly templated complementary strand by said nucleotide (rt-dCTP). In the example, simultaneous or sequential provision of irreversibly terminated (in this case dideoxy) nucleotides ddATP, ddGTP and ddTTP leads to the extension of incorrectly templated complementary strands by one of these irreversibly terminated nucleotides and thus to blockage of any further extension in subsequent cycles. After this extension step, the cycle is completed and a new cycle can be started by deprotection as described above. From the example, it is obvious that only the correct strand would undergo deprotection and further extension.

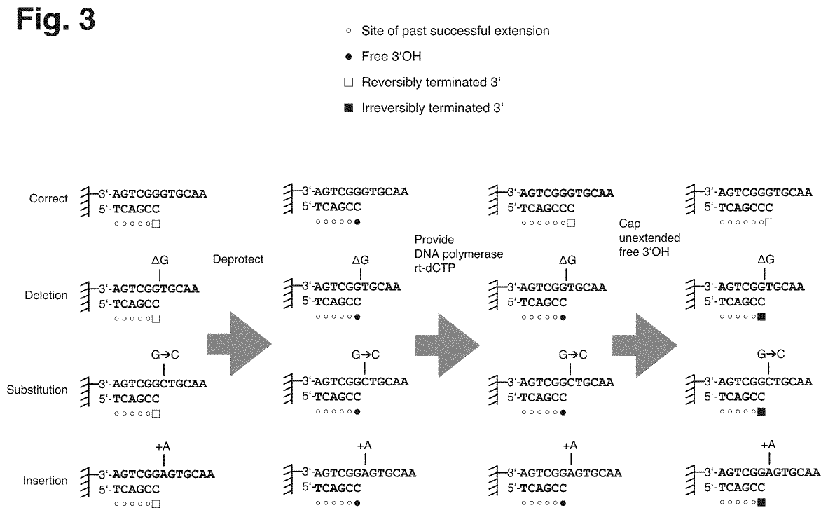

FIG. 3 illustrates is analogous to the cycle described in FIG. 2 but represents another embodiment of the invention, where, instead of irreversible terminators, 3′OH capping is used to terminate incorrectly templated complementary strands [SEQ ID NO:14-18]. In the example shown in FIG. 2, after deprotection of the 3′-O-blocked termini of complementary strands, a 3′-O-blocked rt-CTP and a DNA polymerase are provided as a template ‘G’ is expected at the upcoming position. As a consequence, only the correctly templated strand undergoes extension and bears a reversibly terminated 3′ end, while others expose a 3′OH group. In the next step, a treatment is performed that irreversibly caps said 3′OH groups so that no further extension can occur in subsequent cycles. For example, this may be achieved by means of chemical or enzymatic modification or by incubation with a DNA ligase [SEQ ID NO:11] and a pool of short random oligonucleotides (e.g. 4×N) bearing a dideoxy-3′-end to the effect of hybridising and ligating said oligonucleotides to the free 3′-OH of incorrectly templated complementary strands. After capping is complete, the next cycle of the complementary strand synthesis can be performed, starting again with deprotection.

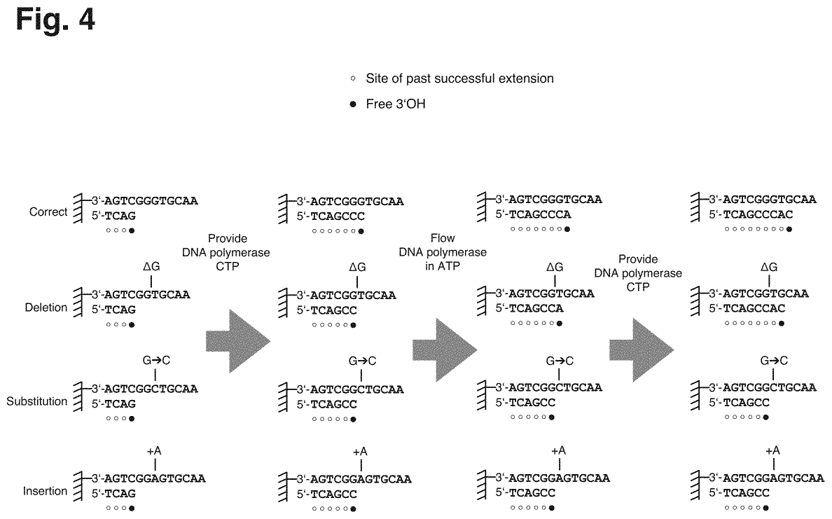

Another embodiment of the present invention is illustrated in FIG. 4, where natural nucleotides (dNTPs) are used for controlled complementary strand synthesis and three cycles of the synthesis process are representatively shown [SEQ ID NO:14-18]. In the first cycle a ‘G’ is expected as the next template base and thus a DNA polymerase is provided together with CTP. In the example, provision of CTP leads to extension of the correctly templated complementary strand by three ‘C’s due to the presence of a homopolymer stretch of three ‘G’s in the template. Likewise, the erroneous strands each get their complementary strands extended by, in this case, two ‘G’s. From the example, as synthesis continues over the next two cycles, it is obvious that the complementary strands for the erroneous templates with the substitution or the insertion will fail to extend and will each lag behind by two bases after two further cycles due to the absence of a correct template base for the provided nucleotide. Moreover, it is obvious that this ‘lag effect’ will become more pronounced over continued cycles, leading to incomplete complementary strand synthesis for substitution or insertion errors whereas deletion errors will not influence the synthesis completeness of the respective complementary strands over continued cycles.

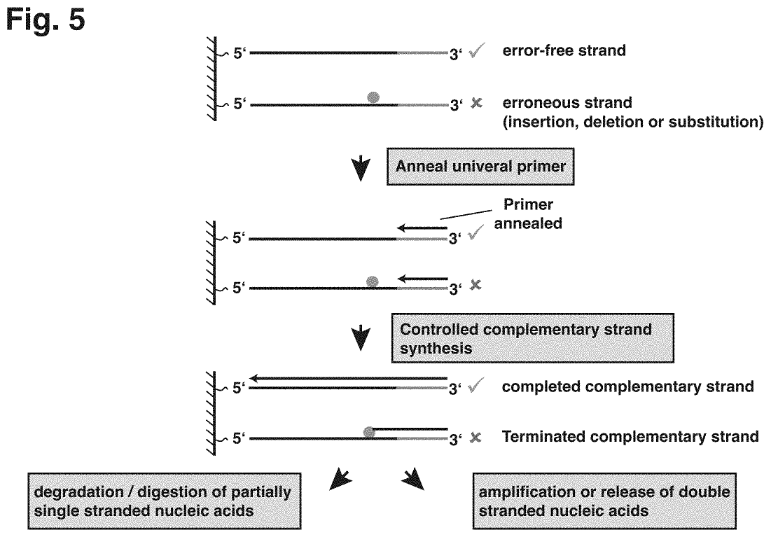

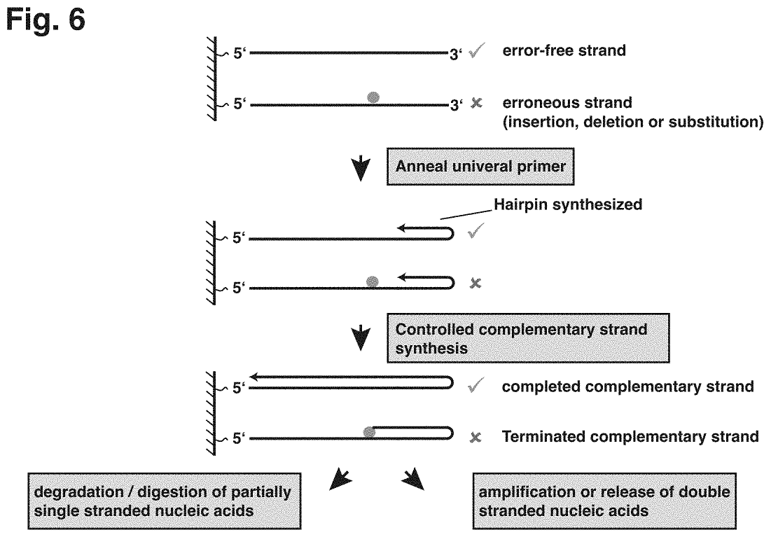

In different embodiments of the invention, the nucleic acids to be interrogated for errors may be immobilized at the 5′- or the 3′-end. For example, 3′-end immobilization is common in phosphoramidite synthesis whereas for enzymatic de novo synthesis 5′-end immobilization is commonly used (illustrated in FIG. 5). While FIGS. 1-4 illustrated different embodiments of the invention described herein based on immobilization at the 3′-end, FIG. 5 shows that 5′-end may as well be applied in analogous manner of previous descriptions herein, with the the starting point of the complementary strand synthesis moved from solid phase-proximal to solid phase-distal. In FIG. 6, an embodiment of the invention is shown, in which 5′-end immobilization of the nucleic acids to be interrogated is used in combination with a terminal hairpin with priming activity for the controlled complementary strand synthesis.

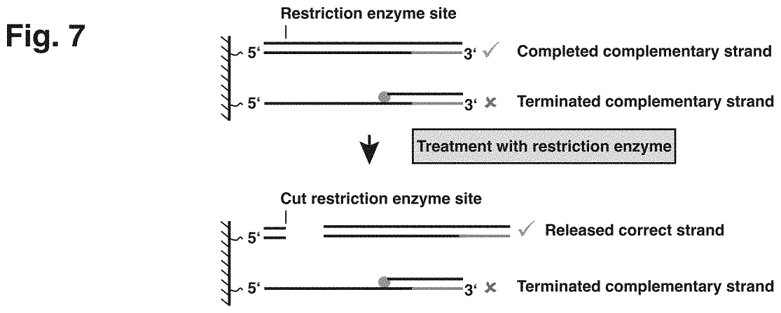

The different embodiments of the invention illustrated in the FIGS. 2, 3 and 4 enable the differentiation of correct nucleic acids based on the progress of complementary strand synthesis. An example of how said differentiation may be exploited is shown in FIG. 7 where a site-specific endonuclease (e.g. Type II or Type IIs restriction enzymes such as NotI [SEQ ID NO:12] or BsaI [SEQ ID NO:13], respectively) with exclusive activity on double stranded nucleic acids is used to release strands that have undergone complete complementary strand synthesis due to the absence of errors in the respective template strands. Released strands may be collected and further processed or used for the intended application. It is not necessary for the accuracy of the complementary strand synthesis and the selective release to be 100% perfect. An enrichment of correct nucleic acids over erroneous nuclei acids already yields significant cost and time advantages.

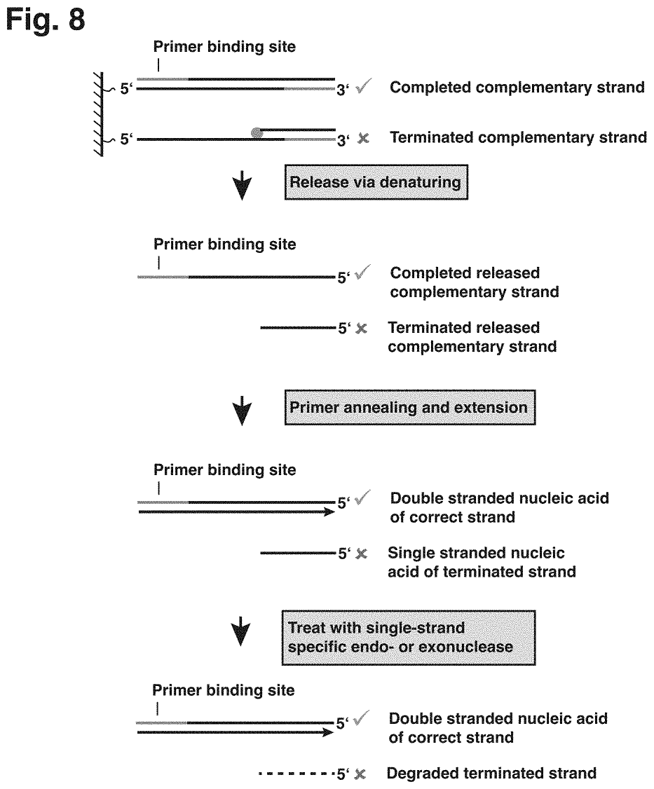

FIG. 8 shows another embodiment the invention, where a release method employing denaturing of the double stranded nucleic acids after complementary strand synthesis is performed. Only non-immobilized strands (which correspond to the complementary strands) will be eluted and may subsequently be annealed to a primer, which can prime the uncontrolled polymerization (i.e. provision of all four natural dNTPs) of a new complementary strand corresponding to the template strand of the first controlled synthesis. Next, a treatment with single-strand specific exo- or endonuclease (e.g. Exonuclease I [SEQ ID NO:6] or Mung Bean Nuclease [SEQ ID NO:9], respectively) may be used to degrade single stranded nucleic acids, which may result from incompletely synthesized complementary strands from the first controlled synthesis and which were hence unable to bind to the primer prior to the uncontrolled polymerization in the previous step.

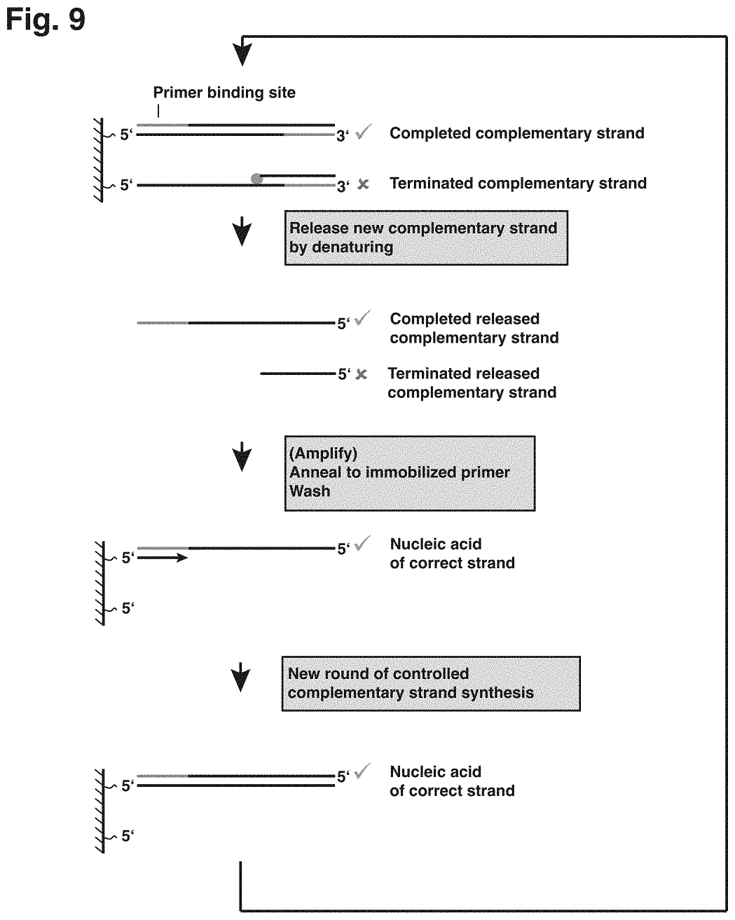

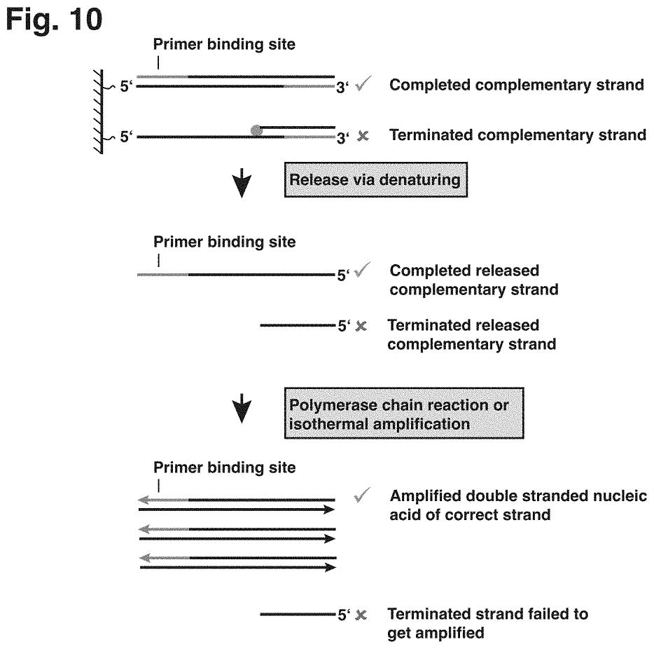

FIG. 9 extends the concept shown in FIG. 8 to a cyclical process that may also be applied to other embodiments of the invention. In the example in FIG. 9, denaturing conditions are used to release complementary strands from a solid support, which may be followed by an amplification process to improve yields of this process. The amplification may for example be performed in solution, in solution in a small fluidic droplet or, in a droplet on a microchip to allow spatial separation from other nucleic acids being synthesized. The amplification may be carried out using polymerase chain reaction or an isothermal amplification process. The optional amplification step may then be followed by binding onto a solid support based on a primer binding site and subsequent controlled complementary strand synthesis. Instead of post-hoc immobilization, said amplification process may already be carried out with immobilized primers (e.g. Bridge PCR or isothermal template walking/‘WildFire’). An example for an amplification-based enrichment for correct nucleic acids is shown in FIG. 10 where it is obvious that terminated strands resulting from the controlled complementary strand synthesis are unable to get amplified due to the lack of the corresponding primer binding site and, for example, the presence of a didexy-3′OH end.

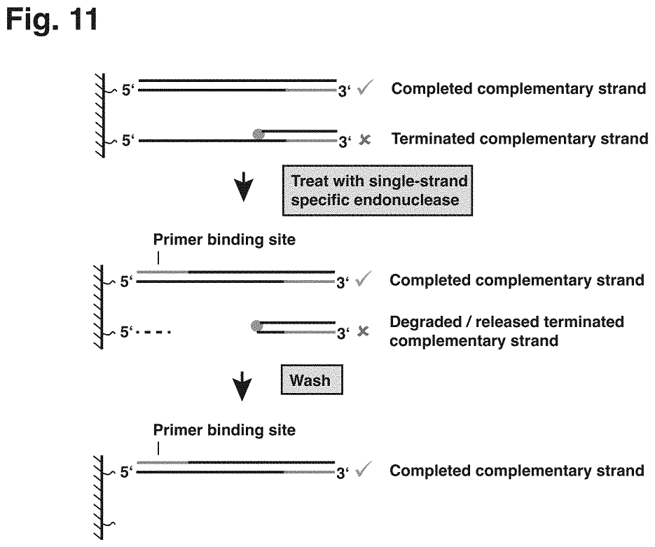

In one embodiment of the invention, instead of selectively releasing correct nucleic acids after controlled complementary strand synthesis, selective detachment of erroneous strands may be performed. FIG. 11 shows how treatment with a single-strand specific endonuclease (e.g. Mung Bean Nuclease [SEQ ID NO:9]) followed by a wash may be used to remove partially single-stranded DNA stemming from incompletely synthesized complementary strands.

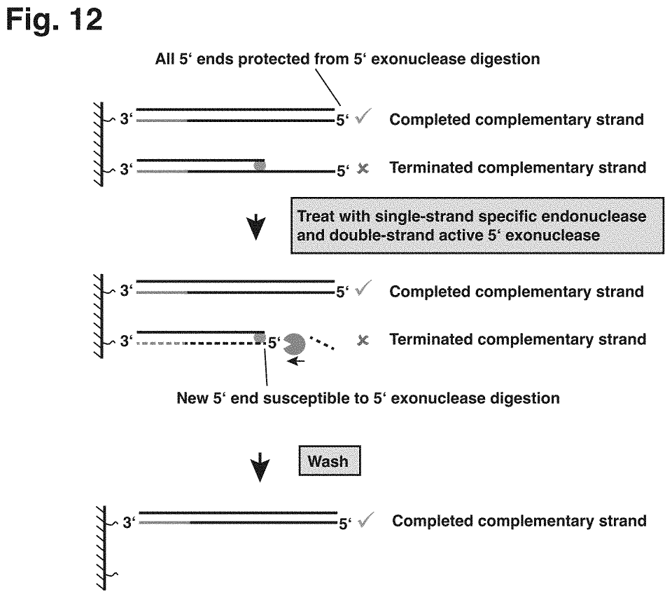

Another method of selective detachment/degradation of erroneous strands is illustrated in FIG. 12 where controlled complementary strand synthesis was carried out on templates with 3′ end immobilisation and where a combination of a single-strand specific endonuclease [SEQ ID NO:9] and a 5′-exonuclease with activity on double-stranded nucleic acids is used for degradation of partially single-stranded DNA, the latter only acting on 5′ ends generated by said single-strand specific endonuclease. For example, before said nuclease treatment all 5′ ends may be non-phosphorylated whereas 5′ ends generated by the single-strand specific endonuclease will be. A 5′ phosphorylation-specific exonuclease [SEQ ID NO:1] may be used to also degrade double-stranded regions of the erroneous nucleic acids, which in turn expose new single-stranded regions, serving as a substrate for the single-strand specific endonuclease [SEQ ID NO:9].

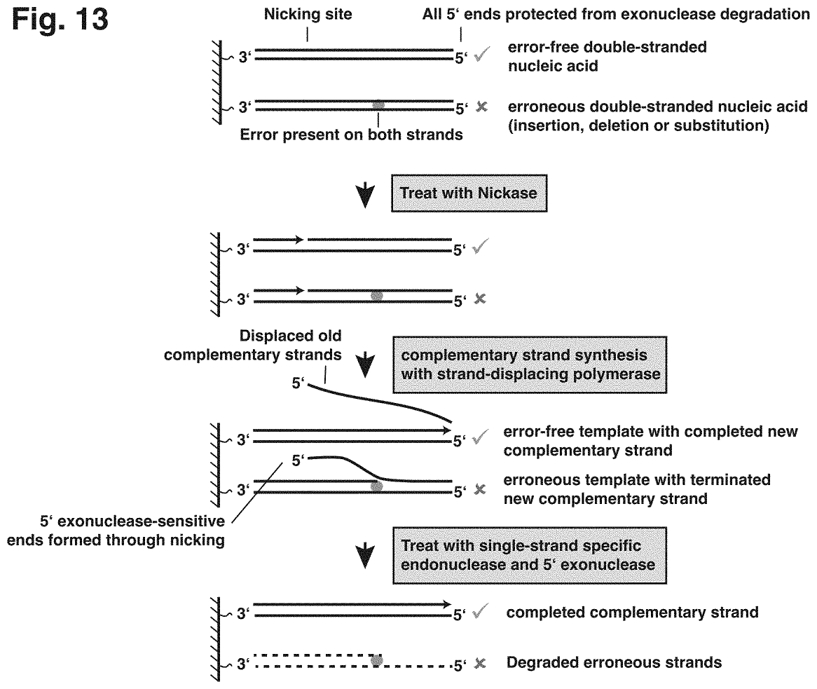

In one embodiment of the present invention, controlled complementary strand synthesis may be initiated on double-stranded nucleic acids. This may be advantageous, for example, if the template strands to be interrogated for errors are expected for form unwanted secondary structures. FIG. 13 shows an example of said process initiated on immobilized double-stranded nucleic acids where the base-by-base complementary strand synthesis is carried out with a polymerase having strand-displacing activity [SEQ ID NO:14] and which is primed through a specifically introduced nick in the non-immobilized strand. After said synthesis process, treatment with a single-strand specific endonuclease [SEQ ID NO:9] and a 5′ exonuclease [SEQ ID NO:1] is carried out in a manner analogous to the example in FIG. 12.

In one embodiment of the invention one or ambiguities may be tolerated or preferred in the synthesis outcome, for example for the purpose of mutagenesis experiments. Multiple ‘versions’ of complementary strands may be synthesized by providing compositions of nucleotides during base-by-base complementary strand synthesis that allow incorporations of more than one type of bases at a certain position.

### REFERENCE TO SEQUENCE LISTING SUBMITTED ELECTRONICALLY

The official copy of the sequence listing is submitted electronically via EFS-Web as an ASCII-formatted sequence listing with a file named “16946376_SL.txt” created on Sep. 29, 2020, and having a size of 52.7 kilobyte, and is filed concurrently with the specification. The sequence listing contained in this ASCII-formatted document is part of the specification and is herein incorporated by reference in its entirety.

1. A method of retrieving at least one nucleic acid from a plurality of template nucleic acids, comprising:
a controlled cyclical synthesis of nucleic acid strands complementary to said template nucleic acids, wherein, at each cycle of said controlled cyclical synthesis, compositions of substrate nucleotides are provided in a way that corresponds to a desired nucleic acid sequence or desired nucleic acid sequence(s) to the effect of selectively extending complementary nucleic acid strands of template nucleic acids comprising said desired sequence(s); and
a subsequent retrieval method comprising:
the selective retrieval of template nucleic acids comprising said desired sequence(s); or
the selective removal of template nucleic acids which do not comprise said desired sequence(s).

2. The method of claim 1, wherein said compositions of substrate nucleotides comprise at least one type of natural nucleotide.

3. The method of claim 1, wherein said compositions of substrate nucleotides comprise at least one type of reversibly terminated nucleotide.

4. The method of claim 1, wherein said compositions of substrate nucleotides comprise at least one type of irreversibly terminated nucleotide.

5. The method of claim 1, wherein said compositions of substrate nucleotides comprise at least one type of natural nucleotide and one type of reversibly terminated nucleotide.

6. The method of claim 1, wherein said compositions of substrate nucleotides comprise at least one type of natural nucleotide and at least one type of irreversibly terminated nucleotide.

7. The method of claim 1, wherein said compositions of substrate nucleotides comprise at least one type of reversibly terminated nucleotide and at least one type of irreversibly terminated nucleotide.

8. The method of claim 1, wherein said compositions of substrate nucleotides comprise at least one type of natural nucleotide, one type of reversibly terminated nucleotide and one type of irreversibly terminated nucleotide.

9. The method of claim 1, wherein said compositions of substrate nucleotides comprise at least one type of reversibly terminated nucleotide and said controlled cyclical synthesis of nucleic acid strands complementary to said template nucleic acids comprises a step to terminate complementary strand synthesis of template nucleic acids that failed to provide a template base complementary to the at least one type of reversibly terminated nucleotide provided in the previous extension step.

10. The method of claim 1, wherein said template nucleic acids are initially single-stranded.

11. The method of claim 1, wherein said template nucleic acids are initially double-stranded.

12. The method of claim 1, wherein at least one strand of each said template nucleic acids is immobilised on a solid surface.

13. The method of claim 1, wherein said subsequent selective retrieval method is enabled by the fully or partially single-stranded nature of template nucleic acids not comprising said desired sequence.

14. The method of claim 1, wherein said subsequent selective retrieval method is enabled by the fully or partially single-stranded nature of template nucleic acids not comprising said desired sequence.

15. The method of claim 1, wherein said subsequent selective retrieval method is enabled by the full or partial absence of a primer binding site in the synthesized complementary strands of template nucleic acids not comprising said desired sequence.

16. The method of claim 1, wherein said controlled cyclical synthesis of nucleic acid strands complementary to said template nucleic acids and said selective retrieval method is performed repeatedly in a recurrent manner.


SEQUENCE LISTING (18 sequences)

SEQ ID NO: 1 - protein, 226 aa, Escherichia phage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 2 - protein, 928 aa, Escherichia coli (strain K12) [sequence omitted]

SEQ ID NO: 3 - protein, 223 aa, Escherichia coli (strain K12)
MDLASLRAQQIELASSVIREDRLDKDPPDLIAGADVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 4 - protein, 157 aa, Enterobacteria phage T4
MLLTGKLYKEEKQKFYDAQNGKCLICQRELNPDVQANHLDHDHELNGPKAGKVRGLLCNLCNAAEGQMKHKFNRSGLKGQGVDYLEWLENLLTYLKSDYTQNNIHPNFVGDKSKEFSRLGKEEMMAEMLQRGFEYNESDTKTQLIASFKKQLRKSLK

SEQ ID NO: 5 - protein, 149 aa, Escherichia phage T7
MAGYGAKGIRKVGAFRSGLEDKVSKQLESKGIKFEYEEWKVPYVIPASNHTYTPDFLLPNGIFVETKGLWESDDRKKHLLIREQHPELDIRIVFSSSRTKLYKGSPTSYGEFCEKHGIKFADKLIPAEWIKEPKKEVPFDRLKRKGGKK

SEQ ID NO: 6 - protein, 475 aa, Escherichia coli (strain K12)
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIV

SEQ ID NO: 7 - protein, 775 aa, Thermococcus species 9 N-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 8 - protein, 277 aa, Escherichia coli (strain K12)
MSNKKQSNRLTEQHKLSQGVIGIFGDYAKAHDLAVGEVSKLVKKALSNEYPQLSFRYRDSIKKTEINEALKKIDPDLGGTLFVSNSSIKPDGGIVEVKDDYGEWRVVLVAEAKHQGKDIINIRNGLLVGKRGDQDLMAAGNAIERSHKNISEIANFMLSESHFPYVLFLEGSNFLTENISITRPDGRVVNLEYNSGILNRLDRLTAANYGMPINSNLCINKFVNHKDKSIMLQAASIYTQGDGREWDSKIMFEIMFDISTTSLRVLGRDLFEQLTSK

SEQ ID NO: 9 - protein, 379 aa, Vigna radiata var. radiata
MPKRRVASVTAAAEEDTLQNHGNNPNEKENSEGNGFFACYLLTSLSPRYKGHTYIGFTVNPRRRIRQHNGEIGCGAFRTKKRRPWEMVLCIYGFPTNVSALQFEWAWQHPVESLAVRKTAVEFKSLSGIANKIKLAYTMLTLSSWQSMNITVNFFSTKYMKHCGGCPSLPAHMKTKTGSLDELPCYSIYGLSEYEDDNVDDVEFDDNNNNTSASGSVPDVSDDLDFPDSPKNQIHGEKISEEFEWIKESEAQEASVNSLSSQEQRLPISSTTPQTTKSSSSSTTTLLQRIEIIEEADFMNVMNKSDSGLIEPAQSDATLAGNTNQTVGSTFVVPHEAEIVDLSTPSPSCRSVLDRKKRRVSSSVTDFIDLTNSPNFVQL

SEQ ID NO: 10 - protein, 509 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 11 - protein, 487 aa, Enterobacteria phage T4
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 12 - protein, 383 aa, Nocardia otitidiscaviarum
MRSDTSVEPEGANFIAEFFGHRVYPEVVSTEAARNDQATGTCPFLTAAKLVETSCVKAETSRGVCVVNTAVDNERYDWLVCPNRALDPLFMSAASRKLFGYGPTEPLQFIAAPTLADQAVRDGIREWLDRGVHVVAYFQEKLGGELSISKTDSSPEFSFDWTLAEVESIYPVPKIKRYGVLEIQTMDFHGSYKHAVGAIDIALVEGIDFHGWLPTPAGRAALSKKMEGPNLSNVFKRTFYQMAYKFALSGHQRCAGTGFAIPQSVWKSWLRHLANPTLIDNGDGTFSLGDTRNDSENAWIFVFELDPDTDASPRPLAPHLEIRVNVDTLIDLALRESPRAALGPSGPVATFTDKVEARMLRFWPKTRRRRSTTPGGQRGLFDA

SEQ ID NO: 13 - protein, 544 aa, Geobacillus stearothermophilus
MGKKAEYGQGHPIFLEYAEQIIQHKEYQGMPDLRYPDGRIQWEAPSNRKSGIFKDTNIKRRKWWEQKAISIGIDPSSNQWISKTAKLIHPTMRKPCKKCGRIMDLRYSYPTKNLIKRIRKLPYVDESFEIDSLEHILKLIKRLVLQYGDKVYDDLPKLLTCKAVKNIPRLGNDLDTWLNWIDSVYIPSEPSMLSPGAMANPPDRLDGFHSLNECCRSHADRGRWEKNLRSYTTDRRAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKENRNALLVINDKIIDKINEIKNILQDIPDEYKLLNEKISEQFNSEEVSDELLRDLVTHLPTKESEPANFKLARKYLQEIMEIVGDELSKMWEDERYVRQTFADLD

SEQ ID NO: 14 - protein, 575 aa, Bacillus phage phi29 [sequence omitted]

SEQ ID NO: 15 - DNA, 12 bp, Artificial Sequence
  synthetic construct
agtcgggtgcaa

SEQ ID NO: 16 - DNA, 11 bp, Artificial Sequence
  synthetic construct
agtcggtgcaa

SEQ ID NO: 17 - DNA, 12 bp, Artificial Sequence
  synthetic construct
agtcggctgcaa

SEQ ID NO: 18 - DNA, 13 bp, Artificial Sequence
  synthetic construct
agtcggagtgcaa